(19) United States
(12) Patent Application Publication
(10) Pub. No.: US 2023/0335256 A1
(43) Pub. Date: Oct. 19, 2023

(54) SUPPORT ON-DEMAND SERVICES (S.O.S) COLLABORATION HUB MOBILE APPLICATION AND COLLABORATIVE (COMMUNITY-WIDE) BEHAVIOR INTERVENTIONS DELIVERY MODEL

(71) Applicant: Chanda L. Spates, Charlotte, NC (US)

(72) Inventor: Chanda L. Spates, Charlotte, NC (US)

(21) Appl. No.: 18/165,255

(22) Filed: Feb. 6, 2023

Related U.S. Application Data
(60) Provisional application No. 63/309,480, filed on Feb. 11, 2022.
(60) Provisional application No. 63/306,959, filed on Feb. 4, 2022.

(51) Int. Cl. G16H 20/70; G16H 80/00; G06Q 50/26

(52) U.S. Cl. CPC G16H 20/70; G06Q 50/26; G16H 80/00

(57) ABSTRACT

The present disclosure provides a mobile hub and behavior interventions strategy method for collaborative and simultaneous delivery of educational and motivational content, community support services, mentoring, behavior and mental health awareness, counseling services, and reinforcement format.

### CROSS-REFERENCE TO RELATED APPLICATIONS

This application claims benefit to U.S. provisional patent application No. 63/306,959, filed Feb. 4, 2022, and U.S. provisional patent application 63/309,480 filed Feb. 11, 2022, the disclosure of which are incorporated herein in their entirety by reference.

### FIELD OF TECHNOLOGY

The following relates generally to a method and process of providing mobile-based behavior intervention strategies.

### BACKGROUND

The disclosure is a mobile application platform and method of delivering and applying behavior intervention strategies such as but not limited to coaching, mentoring, counseling, education, and motivational text, audio, pre-recorded educational content and real-time mentoring and counseling systematically virtually in on centralized platform on which multiple behavior interventions and support services providers are made available and easily accessible in one internet mobile application center.

Children, teens, and young adults ages 14-24 lacking access to a positive male role model have been shown to be significantly more likely to have behavioral, emotional, and criminal behaviors those of the same population that have access to a positive male role model.

Conventional methods of solving this challenge are labor-intensive and greatly reduce the ability to reach the majority of patients or clients needing services.

### BRIEF SUMMARY

The disclosure is a mobile application hub and behavior interventions strategy method for collaborative and simultaneous delivery of educational and motivational content, community support services, mentoring, behavior and mental health awareness, counseling services, and reinforcement format. The hub consists of specific educational and motivational content formatting of a virtual, on-demand, pre-recorded video library, and audio programming.

Under certain circumstances, an embodiment includes a method of providing interactive counseling services to a user via a computer controller in real-time during a virtual counseling session. The method includes receiving at the computer controller, a first request from the user for interactive counseling services, presenting to the user via a display coupled to the computer controller, (i) a generic message related to the user’s request, and (ii) a menu of topics responsive to the requested counseling. The menu includes at least one of video libraries, radio content, and streaming services associated with the requested counseling, the video libraries, radio content, and streaming services simulating advice from an authority figure associated with the user. The method also includes providing the user access to emergency treatment services when the generic message and the menu of topics are presented, the access to emergency treatment services being provided external to the virtual counseling session and matching the user with a human counselor responsive to a second request from the user.

Embodiments include a system that provides on-demand support, coordinated community engagement and awareness activities, and mobile accessibility portals. The system ultimately provides multiple mentoring, behavior and mental health service providers to create a macro-level collaborative method for addressing a wide range of shared social problems within a community, city, county, municipality, or national level.

The virtual application and delivery of counseling, coaching, and behavior modeling increases access to the services in a uniquely real time there are the patients’ or clients’ crisis is occurring. Displaces coaching and counseling at the patient’s immediate access and when needed the most. The access to positive male role models at the exact point of crisis is unique and enables improved outcomes for behavior intervention strategies.

Additional features, modes of operations, advantages, and other aspects of various embodiments are described below with reference to the accompanying drawings. It is noted that the present disclosure is not limited to the specific embodiments described herein. These embodiments are presented for illustrative purposes only. Additional embodiments, or modifications of the embodiments disclosed, will be readily apparent to persons skilled in the relevant art(s) based on the teachings provided herein.

### BRIEF DESCRIPTION OF THE DRAWINGS

Illustrative embodiments may take form in various components and arrangements of components. Illustrative embodiments are shown in the accompanying drawings, throughout which like reference numerals may indicate corresponding or similar parts in the various drawings. The drawings are only for purposes of illustrating the embodiments and are not to be construed as limiting the disclosure. Given the following enabling description of the drawings, the novel aspects of the present disclosure should become evident to a person of ordinary skill in the relevant art(s).

FIGS. 1-22 provide various exemplary illustrations of embodiments of the disclosure.

FIG. 23 illustrates a mobile app hub flow in accordance with the embodiments.

FIG. 24 illustrates a reactive browsing carousel menu in accordance with the embodiments.

FIG. 25 illustrates upcoming sessions and functions in accordance with the embodiments.

FIG. 26 illustrates key Sid menu functions in accordance with the embodiments.

FIG. 27 illustrates an exemplary computer system on which embodiments of the present disclosure may be practiced.

### DETAILED DESCRIPTION

While the illustrative embodiments are described herein for particular applications, it should be understood that the present disclosure is not limited thereto. Those skilled in the art and with access to the teachings provided herein will recognize additional applications, modifications, and embodiments within the scope thereof and additional fields in which the present disclosure would be of significant utility.

Embodiments of the disclosure increase accessibility to independent community support programs, behavior and mental health services and increases the effectiveness of those programs and support services by the addition education, motivational, and reinforcement activities, coordination of collaborative community engagement.

Embodiments provide a hub based system which allows it to be customized for specific markets, youth, military, and transitioning ex-offenders. By way of example, a mobile application embodiment allows users to speak to, ask a question to and then provides a copy of the written behavioral health/mental health response. The application has integrated radio streaming and character-building content and an optional telehealth chat room for secure real-time counseling.

The intervention works by creating a platform by which application users are able to learn about services, research for answers to common questions and topics, through self service in-demand video answer library. In addition, Application users are able to access multiple behavior and mental health professional and mentoring services through pre-recorded online video, webinar, trainings on relevant mental health and educational topics.

The disclosure connects the app user with access to multiple independent service providers with one application as the point of entry to many services and providers. The hub utilizes a content formatting and reinforcement process, or behavior intervention model, as outlined above and below.

Disclosure users are able to sign into the mobile application hub to search and play educational content by keyword search on thousands of topics. The user is able to listen to audio in the form streaming radio, on-demand blog, webinar, and training on related support topics. The User is able to save and replay favorite video and educational content. Additional users are able to proactively seek support by chatting, video conferences, or sign into selected independent behavior and mental health support services via portal for real time live mental health support.

Other tier subscription users such as mental health providers, parents, teachers, and agencies are able auto access additional content including lesion plans educational curriculum, patent discussion guides, to aid in providing support for their subscribed clients and app users.

Reports from aggregate data collected from the mobile application hub may be used by subscribed school districts, community leaders, local, state, and government agencies to help guide the types of behavior interventions needed based on app user trends for topics and search queries by location and age. This aggregate data is an essential tool for improved detection to response time and de-escalation of areas of concern as the hub acts as a pulse of a community, this increasing overall effectiveness of community resources and support programs.

The application may be used for many customizable applications including but not limited to mentoring, behavioral health, depression and suicide prevention, weight loss support, case management, and self-help industry. The program may be customized for client specifications to link related services such as real estate portal to connect realtors, lending institutions, staging professionals, and leasing agencies.

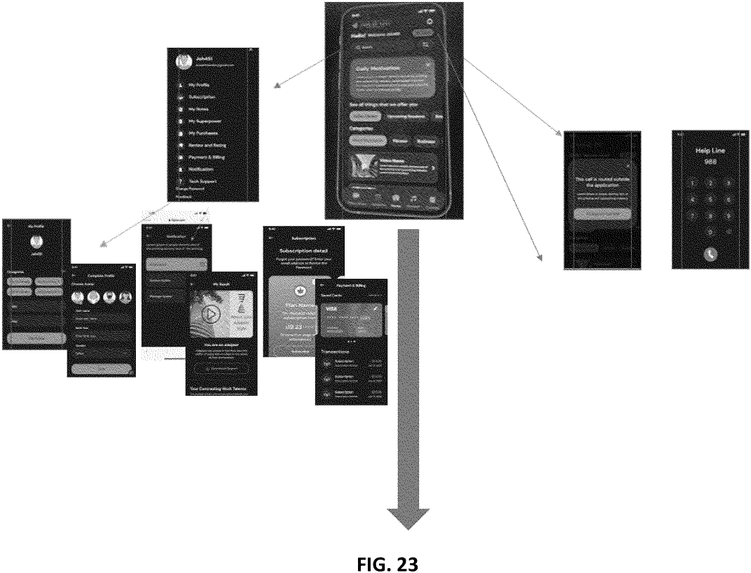

FIG. 23 illustrates a mobile app hub flow in accordance with the embodiments. As depicted in FIG. 23, users may upload a picture/image/icon/ or select an avatar (setting for minor users under age 18 by default based on birth month and year provided in profile set up.

The process complies with HIPAA minor privacy requirements as of submission date. Minor’s full names and other Protected Information are not captured for minor users. Survey results - Type of survey is customizable based on client or application specification. The survey shown is for illustration purposes only.

System users may customize and select settings options including notification frequency, subscription level (packages), and submit billing information from within the application. The system will accept credit, debit, and health insurance card entries based on client specifications and integration with telehealth platform.

The system proactively prompts user for opportunities to make help call based on preset at-risk behavior thresholds set by client or application protocol ex. 911,988, crisis, nurse line, employee assistance, or client designated help/support line. Other calls prohibited or blocked from within app.

Ex: repeated launch of high risk library topics and/or more than present 15-30-45 minutes within suicide video library topic may prompt user for option to launch help call.

Proactive help dialing may be turned on or off based on client specifications. The system reads subscribers signup group for determining appropriate/assigned call line. Call to the National Suicide Hotline shown in illustration is for illustration of this feature, however the help number may be determined by application the client.

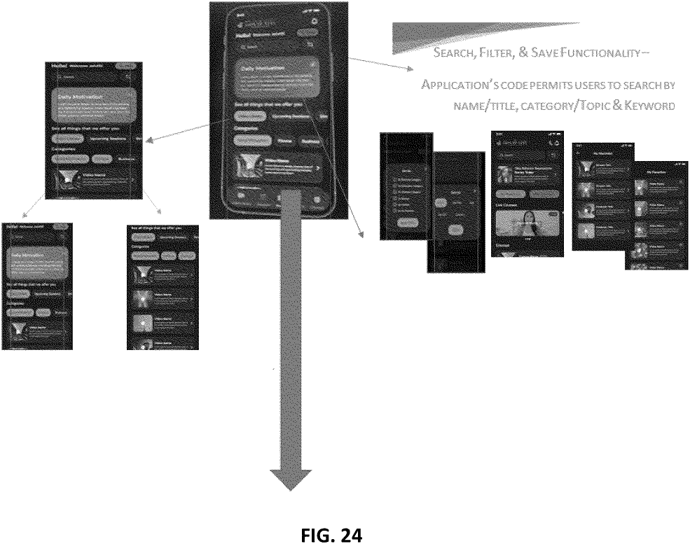

FIG. 24 illustrates a reactive browsing carousel menu. In FIG. 24, code permits user to browse various (client determined) video library topics, categories, and content. The system highlights current browsing category or categories being displayed.

Carousel browsing menu scrolls left or right. The system displays associated and corresponding video topics as browsing menu selections change. Upcoming sessions, streams, and courses sections may be accessed via the reactive carousel menu. Upcoming sessions routine reads appointments from integrated telehealth or appointments calendar based if telehealth integration is requested for application or client request. If selected, code will display previous and upcoming sessions. A user may enter notes however clinical notes do not display within application.

The system launches keyword search against application’s content video, courses, and event n content, videos, and course titles and returns a list of matching video, course, blogs, or related content found. The system may rank and sort search results by relevance to key word search term/description.

The system matches and displays streaming radio and podcast streams based on the user’s selection. The code assigned selected titles, videos, and courses to watch list (titles not yet viewed or to watch later and favorites (frequent access) options.

Daily motivational quote. The system displays a daily motivational quote. Applications within the system pull from client or application’s pre-selected quotes based on client or application reference.

The system displays a different quote each day at random or may display quotes as specified on specific date, holiday, or specified order. Applications display a different quote each day at random or may display quotes as specified on specific date, holiday, or specified order.

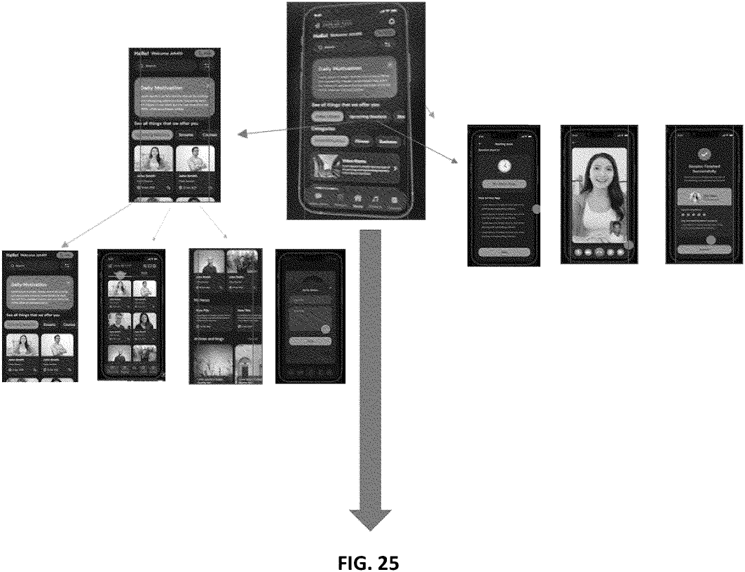

FIG. 25 illustrates upcoming sessions and functions in accordance with the embodiments. The system displays past and upcoming sessions based on integrated appointment calendar (Telehealth).

The system complies with HIPAA Telehealth compliant meeting platform integration and telehealth portals. Embodiments offer in app telehealth video and audio conferencing. The system also permits user to enter notes. Therapist, counselor, and mentor notes are not entered into the mobile application, however, may be entered via telehealth portal integration.

The system supports individual, and group virtual sessions (family therapy). Meeting waiting room permits therapist control of meeting start and breakout rooms. The embodiments support individual, and group virtual sessions (family therapy). Meeting waiting room permits therapist control of meeting start and breakout rooms.

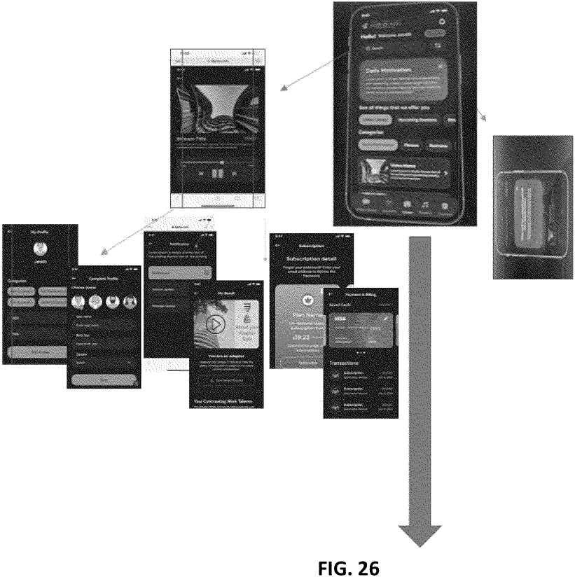

FIG. 26 illustrates key Sid menu functions in accordance with the embodiments. As illustrated in FIG. 26, users may upload a picture/image/icon/ or select an avatar (setting for minor users under age 18 by default.

The embodiments comply with HIPAA minor privacy requirements as of submission date. Minor’s full names and other Protected Information are not captured for minor users.

Survey results - type of survey is customizable based on client or application specification. The survey shown is for illustration purposes only. Users of the embodiments may customize and select settings options including notification. The embodiments proactively prompt user for opportunities to make help call based on preset at-risk behavior thresholds set by client or application protocol. Proactive help dialing may be turned on or off based on client specifications.

By way of example, calls to the National Suicide Hotline shown in illustration is for illustration of this feature, however the help number may be determined by application client request. Application components display a daily motivational quote. The system pulls from 90-366 pre-selected quotes based on client or application reference. A different quote may be displayed each day or may be set to display quote randomly if the 90 quote options is selected.

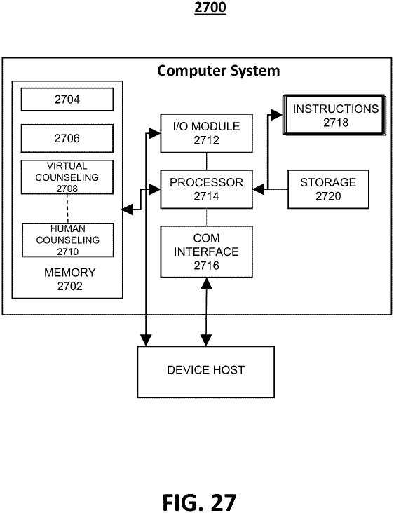

FIG. 27 illustrates a computer system 2700 that may be an application-specific hardware, software, and firmware implementation on which embodiments of the present disclosure may be implemented. The computer system 2700 can include a processor 2714 configured to executed one or more, or all of the blocks of the method 200, the method 300, or the functions of the exemplary system described above.

The processor 2714 can have a specific structure imparted to the processor 2714 by instructions stored in a memory 2702 and/or by instructions 2718 fetchable by the processor 2714 from a storage medium 2720. The storage medium 2720 can be remote and communicatively coupled to the computer system 2700. Such communications can be encrypted.

The computer system 2700 can be a stand-alone programmable system, or a programmable module included in a larger system. For example, the computer system 2700 can be included in hardware associated with the POS device 114 connected to the computer system 2700. The computer system 2700 may include one or more hardware and/or software components configured to fetch, decode, execute, store, analyze, distribute, evaluate, and/or categorize information.

The processor 2714 may include one or more processing devices or cores (not shown). In some embodiments, the processor 2714 may be a plurality of processors, each having either one or more cores. The processor 2714 can execute instructions fetched from the memory 2702, i.e., from one of memory modules 2704, 2706, 2708, or 2710. Alternatively, the instructions can be fetched from the storage medium 2720, or from a remote device connected to the computer system 2700 via a communication interface 2716. Furthermore, the communication interface 2716 can also interface with computer systems within a computer system of the host. An input/output (I/O) module 2712 may be configured for additional communications to or from associated remote systems.

Without loss of generality, the storage medium 2720 and/or the memory 2702 can include a volatile or non-volatile, magnetic, semiconductor, tape, optical, removable, non-removable, read-only, random-access, or any type of non-transitory computer-readable computer medium. The storage medium 2720 and/or the memory 2702 may include programs and/or other information usable by processor 2714. Furthermore, the storage medium 2720 can be configured to log data processed, recorded, or collected during the operation of computer system 2700.

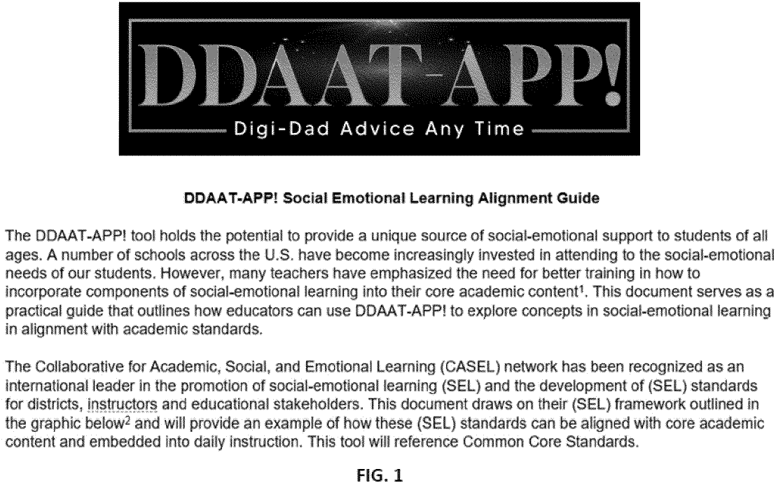

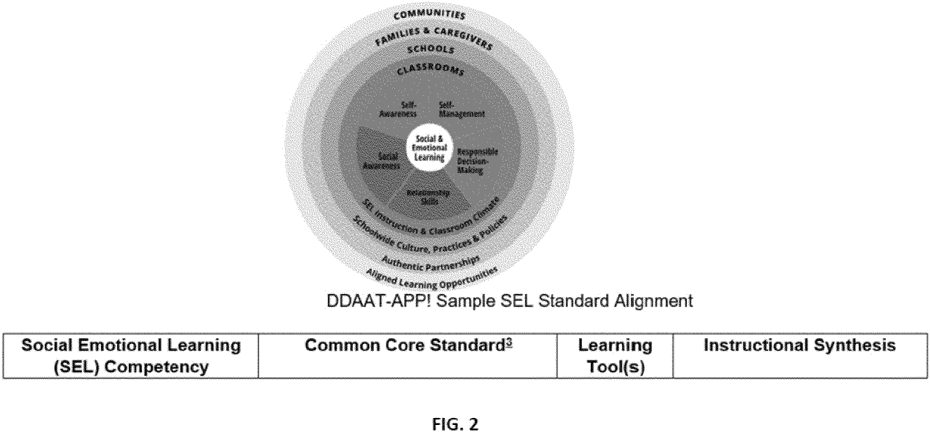

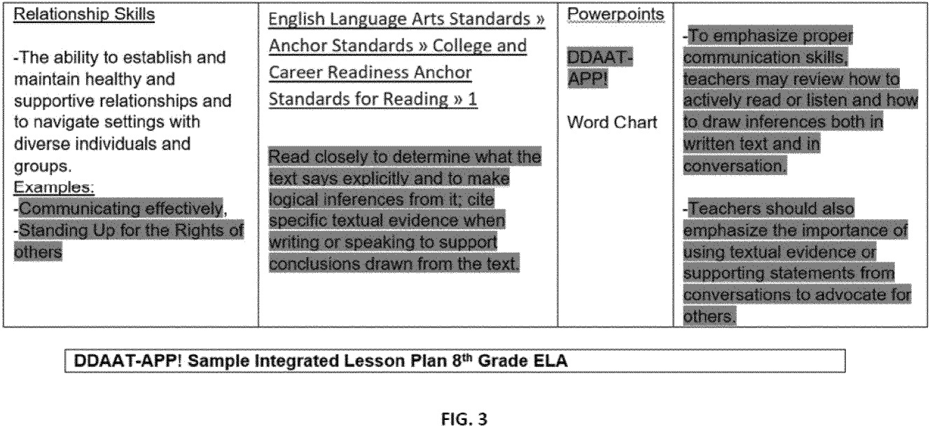

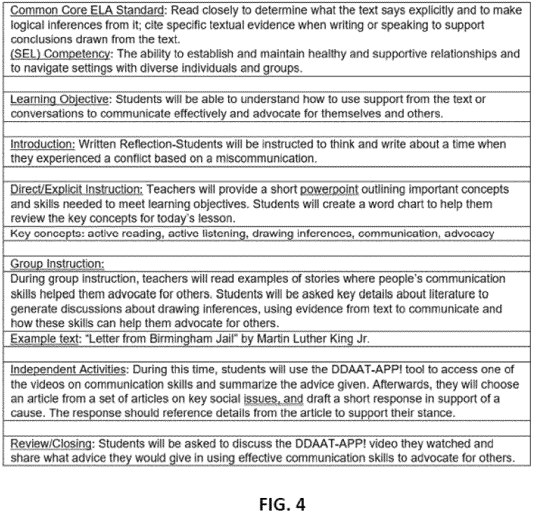

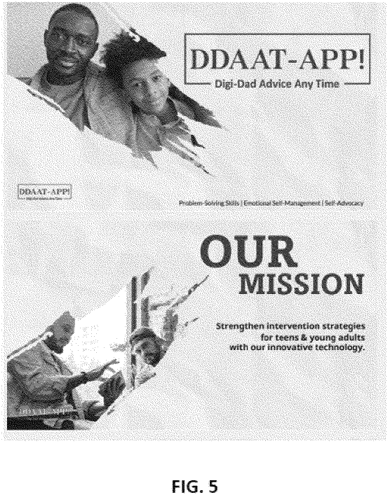

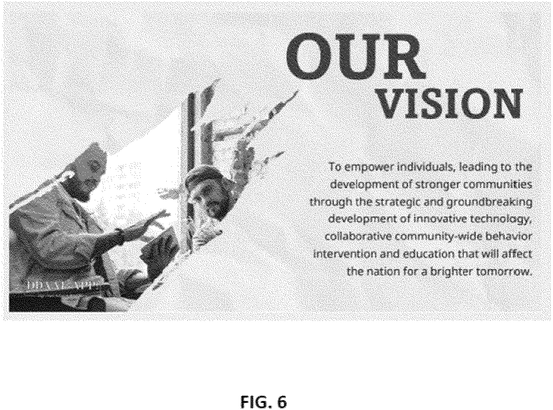

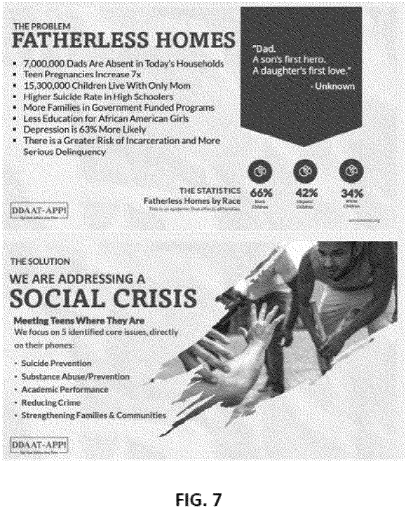

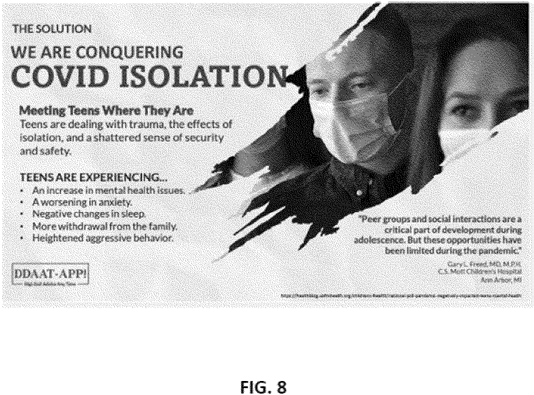

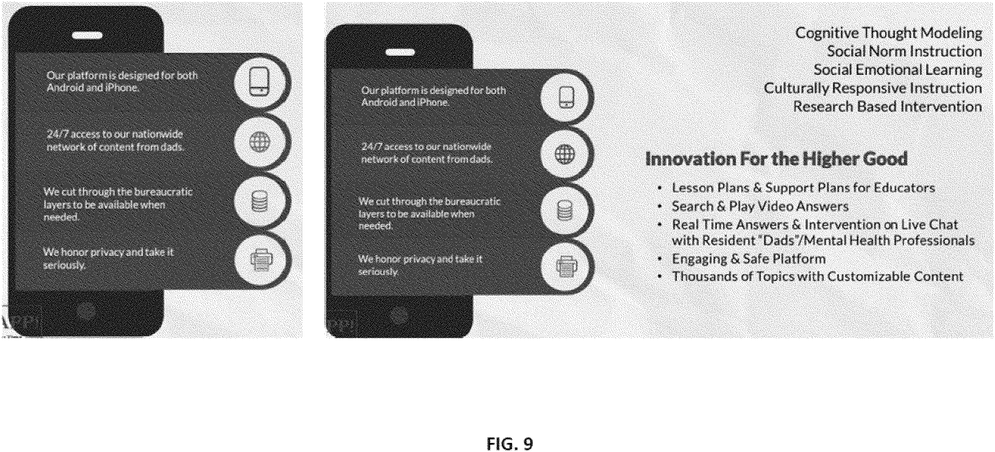

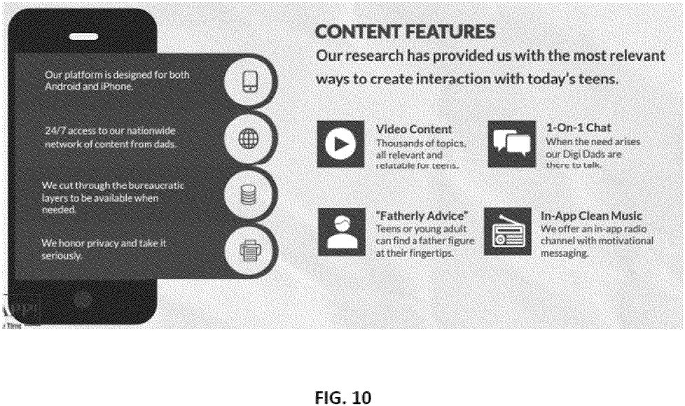

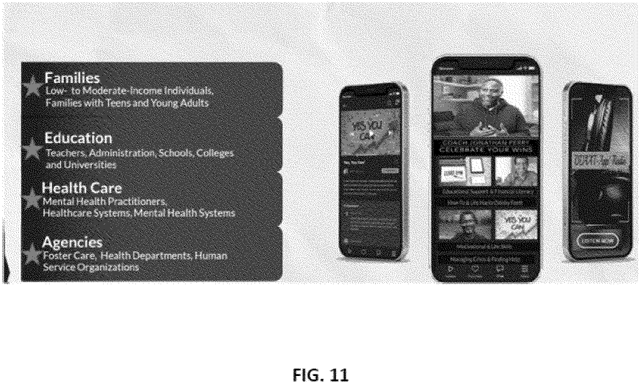

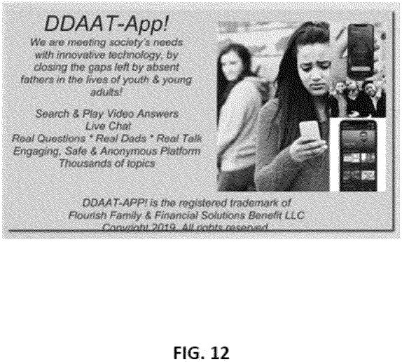

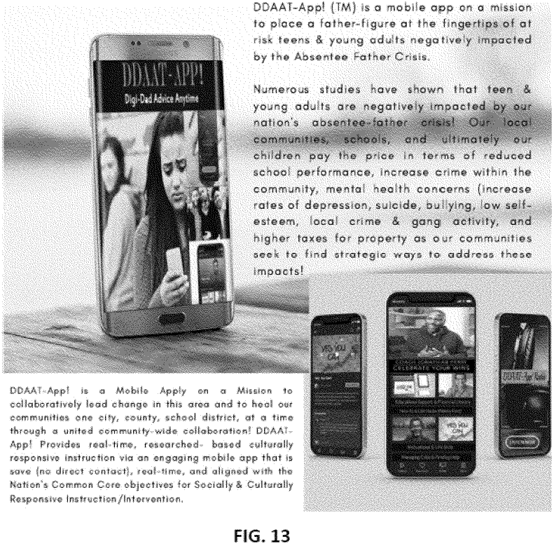

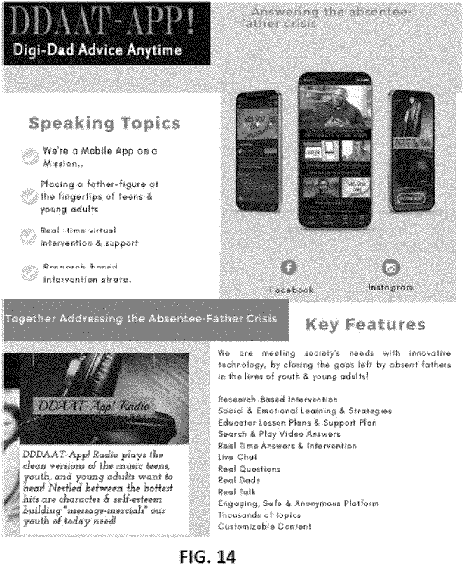

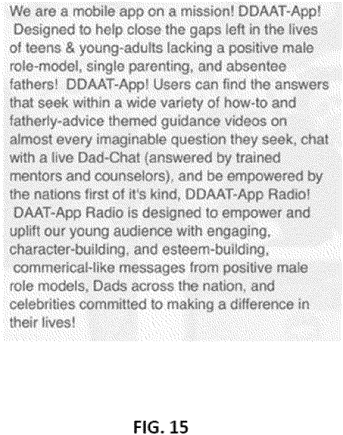

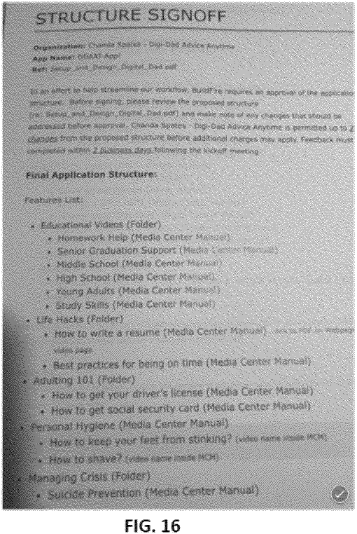

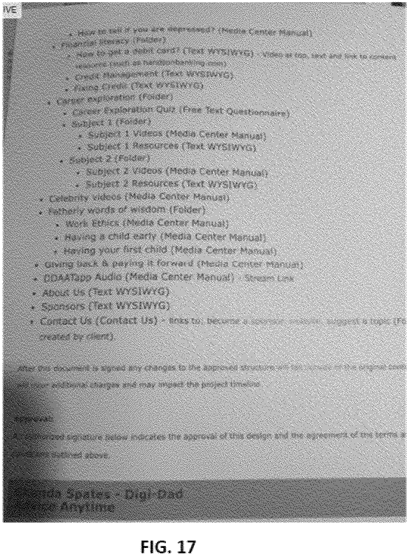

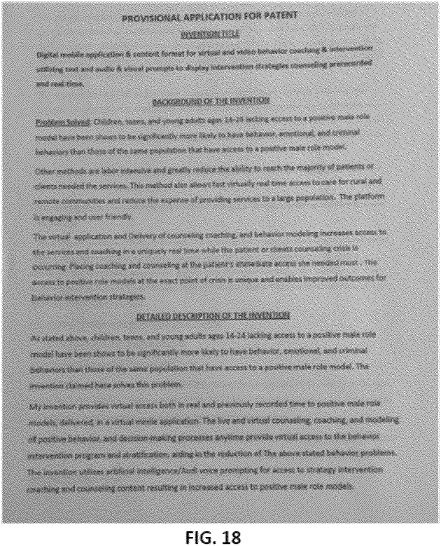

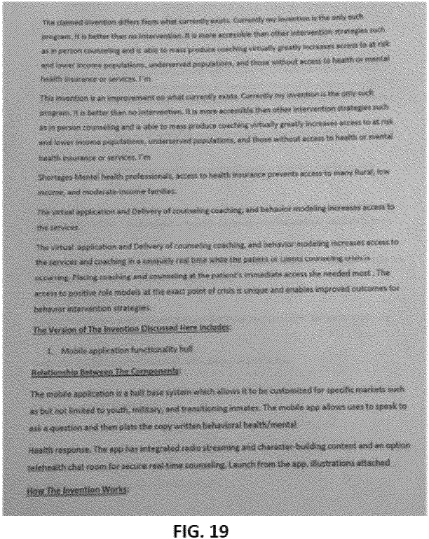

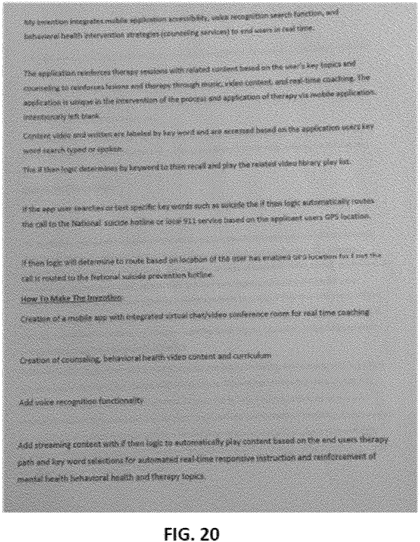

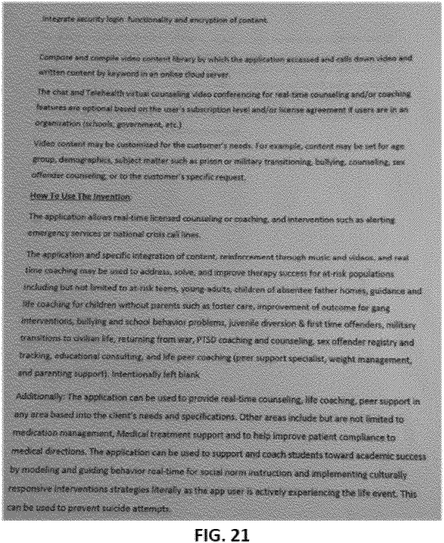

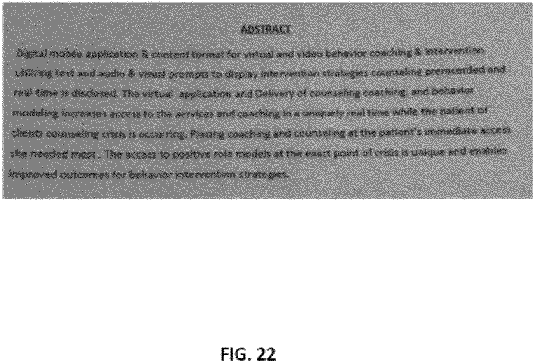

The data may be time-stamped, location-stamped, cataloged, indexed, encrypted, and/or organized in a variety of ways consistent with data storage practices. By way of example, the memory module 2708 can store instructions for executing the virtual counseling session and the memory module 2710 can include a routine for matching user with real-time human counseling. The instructions embodied in these memory modules can cause the processor 2714 to perform certain operations consistent with the functions described in FIGS. 1-27 above.

The description herein is provided to enable a person skilled in the art to make or use the disclosure. Various modifications to the disclosure will be apparent to those skilled in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

1. A method of providing interactive counseling services to a user via a computer controller in real-time during a virtual counseling session, the method comprising:
receiving at the computer controller, a first request from the user for interactive counseling services;
presenting to the user via a display coupled to the computer controller, (i) a generic message related to the user’s request, and (ii) a menu of topics responsive to the requested counseling;
wherein the menu includes at least one of video libraries, radio content, and streaming services associated with the requested counseling, the video libraries, radio content, and streaming services simulating advice from an authority figure associated with the user;
providing the user access to emergency treatment services when the generic message and the menu of topics are presented, the access to emergency treatment services being provided external to the virtual counseling session; and
matching the user with real-time human counseling responsive to a second request from the user.

2. The method of claim 1, wherein the computer controller includes at least one of a mobile phone, a laptop computer, a desktop computer, and a tablet computer.

3. The method of claim 1, wherein the counseling services are associated with a hub of different types of services.

4. The method of claim 1, wherein the authority figure may include at least one of a parent, mentor, and trusted agent.

5. A non-transitory computer readable medium having stored thereon computer executable instructions that, if executed by a computing device, cause the computing device to perform a method of providing interactive counseling services to a user via a computer controller in real-time during a virtual counseling session, the method comprising:
receiving at the computer controller, a first request from the user for interactive counseling services;
presenting to the user via a display coupled to the computer controller, (i) a generic message related to the user’s request, and (ii) a menu of topics responsive to the requested counseling;
wherein the menu includes at least one of video libraries, radio content, and streaming services associated with the requested counseling, the video libraries, radio content, and streaming services simulating advice from an authority figure associated with the user;
providing the user access to emergency treatment services when the generic message and the menu of topics are presented, the access to emergency treatment services being provided external to the virtual counseling session; and
matching the user with real-time human counseling responsive to a second request from the user.
